(19) United States
(12) Patent Application Publication
(10) Pub. No.: US 2021/0052885 A1
(43) Pub. Date: Feb. 25, 2021

(54) IMPLANTABLE ELECTRODE DEVICE, MEDICAL DEVICE OR SYSTEM THEREOF SUCH AS NEUROSTIMULATOR, AND METHOD THEREOF

(71) Applicant: Norbert Kaula, Arvada, CO (US)

(72) Inventor: Norbert Kaula, Arvada, CO (US)

(21) Appl. No.: 17/001,571

(22) Filed: Aug. 24, 2020

Related U.S. Application Data
(60) Provisional application No. 62/890,569, filed on Aug. 22, 2019.

(51) Int. Cl. A61N 1/05

(52) U.S. Cl. CPC A61N 1/0556; A61N 1/0558; A61N 1/0553

(57) ABSTRACT

The present invention provides an implantable electrode device, a medical device/system thereof, and a diagnostic and/or therapeutic method using the medical device/system. The implantable electrode device includes a flexible non-conductive flap, one or more electrodes integrated with the flap, and one or more wires embedded within the flap and connected to the one or more electrodes. The invention exhibits numerous technical merits such as stable fixation of the electrode to a biological target in a patient.

### CROSS-REFERENCE TO RELATED APPLICATIONS

This application expressly claims the benefits and priority based on U.S. Provisional Application No. 62/890,569 filed Aug. 22, 2019, which is incorporated herein by references as if set forth in its entirety.

### FIELD OF THE INVENTION

The present invention provides an implantable electrode device, a medical device or a medical system thereof, and a diagnostic and/or therapeutic method using the medical device or system. Particularly, an embodiment of the present invention relates to a medical system and a medical device comprising a flap-shaped implantable lead, and a method and an application thereof in nerve stimulation. Although the invention will be illustrated, explained and exemplified with an implantable lead to stimulate anterior and posterior spinal nerve components, it should be appreciated that the present invention can also be applied otherwise and/or in other fields.

### BACKGROUND OF THE INVENTION

Various implantable electrodes used in medical devices include cuff, percutaneous or thin-film based electrodes. Advantageously, the present invention provides a novel electrode device that can stably affixed to a biological target in a patient such as a nerve. Preferably, the novel electrode device can be, but is not limited to, an implantable lead or electrode.

### SUMMARY OF THE INVENTION

One aspect of the present invention provides an implantable electrode device comprising a flexible non-conductive flap, one or more electrodes integrated with the flap, and one or more wires embedded within the flap and connected to the one or more electrodes. The implantable electrode device is configured to wrap around one, two or more biological targets, so that one of the electrodes can electrically and stably contact one of the biological targets. A center of the flap can be pulled up between two biological targets such as two nerve segments, creating two circular like electrodes around each of the two biological targets such as nerve segments.

Another aspect of the invention provides a medical device or a medical system comprising the implantable electrode device as summarized above.

Still another aspect of the invention provides a diagnostic and/or therapeutic method comprising (i) providing a medical device or a medical system as summarized above, (ii) wrapping the implantable electrode device as summarized above around one, two or more biological targets in a patient, so that one of the electrodes can electrically and stably contact one of the biological targets.

The above features and advantages and other features and advantages of the present invention are readily apparent from the following detailed description of the best modes for carrying out the invention when taken in connection with the accompanying drawings.

### BRIEF DESCRIPTION OF THE SEVERAL VIEWS OF THE DRAWINGS

The present invention is illustrated by way of example, and not by way of limitation, in the figures of the accompanying, drawings and in which like reference numerals refer to similar elements. All the figures are schematic and generally only show parts which are necessary in order to elucidate the invention. For simplicity and clarity of illustration, elements shown in the figures and discussed below have not necessarily been drawn to scale. Well-known structures and devices are shown in simplified form, omitted, or merely suggested, in order to avoid unnecessarily obscuring the present invention.

FIG. 1A schematically shows a medical device or system in accordance with an exemplary embodiment of the present invention.

FIG. 1B is a flow chart of the diagnostic and/or therapeutic method in accordance with an exemplary embodiment of the present invention.

FIG. 2 shows a flap wrapped around (partially or completely) an anterior nerve bundle and a posterior nerve bundle in accordance with an exemplary embodiment of the present invention.

FIG. 3 illustrates electrodes on both sides of the flap are required to wrap around (partially or completely) an anterior nerve bundle and a posterior nerve bundle in accordance with an exemplary embodiment of the present invention.

FIG. 4 demonstrates an implantable electrode device including microelectrode arrays and suture holes in accordance with an exemplary embodiment of the present invention.

FIG. 5 depicts some details of a stimulation contact in accordance with an exemplary embodiment of the present invention.

FIG. 6 illustrates the relationship between contacting areas in the flap and corresponding contacting areas in the nerves in accordance with an exemplary embodiment of the present invention.

FIG. 7 schematically shows a manner how two flaps are wrapped around (partially or completely) an anterior nerve bundle and a posterior nerve bundle in accordance with an exemplary embodiment of the present invention.

FIG. 8 schematically shows a manner how a single flap is wrapped around (partially or completely) an anterior nerve bundle and a posterior nerve bundle in accordance with an exemplary embodiment of the present invention.

### DETAILED DESCRIPTION OF THE PREFERRED EMBODIMENT

In the following description, for the purposes of explanation, numerous specific details are set forth in order to provide a thorough understanding of the present invention. It is apparent, however, to one skilled in the art that the present invention may be practiced without these specific details or with an equivalent arrangement.

Where a numerical range is disclosed herein, unless otherwise specified, such range is continuous, inclusive of both the minimum and maximum values of the range as well as every value between such minimum and maximum values. Still further, where a range refers to integers, only the integers from the minimum value to and including the maximum value of such range are included. In addition, where multiple ranges are provided to describe a feature or characteristic, such ranges can be combined.

It is also to be understood that the terminology used herein is for the purpose of describing particular embodiments only, and is not intended to limit the scope of the invention. For example, when an element is referred to as being “on”, “connected to”, or “coupled to” another element, it can be directly on, connected or coupled to the other element or intervening elements may be present. In contrast, when an element is referred to as being “directly on”, “directly connected to”, or “directly coupled to” another element, there are no intervening elements present.

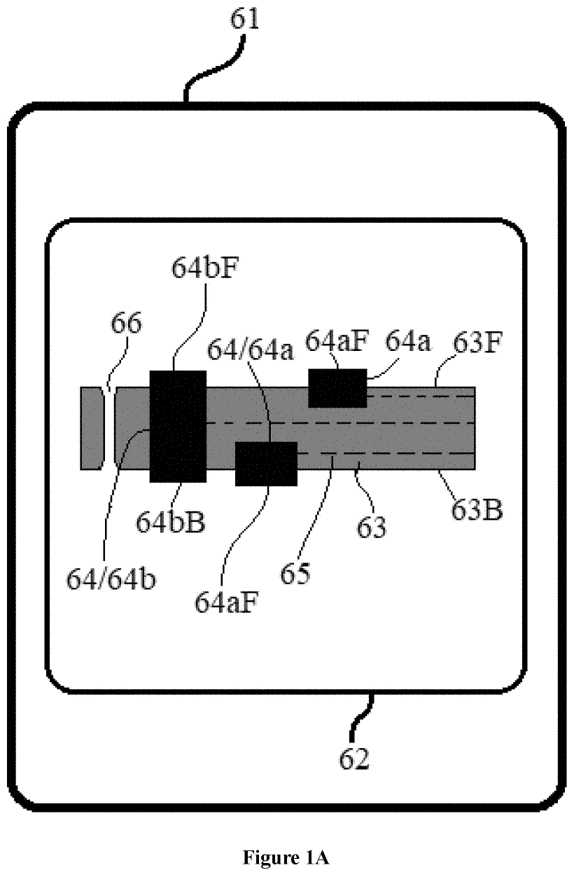

As shown in FIG. 1A, a medical device or system 61 includes an implantable electrode device or lead 62. Implantable electrode device 62 includes a flexible non-conductive flap 63, one or more electrodes 64 integrated with the flap, and one or more wires 65 (represented as broken lines) embedded within the flap 63 and connected to the one or more electrodes 64. The implantable electrode device 62 is configured to wrap around one, two or more biological targets (not shown), so that one of the electrodes 64 can electrically and stably contact one of the biological targets.

The flap 63 may have a front side 63F and a back side 63B. One of the electrodes 64a may have only one flat contacting surface 64aF that can electrically contact one of the biological targets. The contacting surface 64aF may be on the front side of the flap 63F, or on the back side of the flap 63B. Alternatively, one of the electrodes 64b may have only two flat contacting surfaces (64bF, 64bB) that can electrically contact one of the biological targets One of the two contacting surfaces 64bF may be on the front side of the flap 63F, and another one 64bB is on the back side of the flap 63B, and vice versa.

In preferred embodiments, the flap 63 comprises one or more suture holes 66 (typically through holes) for stitching the flap 63 around the one, two or more biological targets.

FIG. 1B is a flow chart of the diagnostic and/or therapeutic method in an embodiment of the invention. The method includes step (i) providing a medical device/system as described above and illustrated in FIG. 1A; and step (ii) wrapping the implantable electrode device as described above and illustrated in FIG. 1A around one, two or more biological targets in a patient, so that one of the electrodes can electrically and stably contact one of the biological targets (e.g. one or more nerves).

In some embodiments where the flap comprises one or more suture holes for stitching the flap around the one or more nerves, the method may further include a step (iii) stitching the flap around the one or more nerves through the one or more suture holes, as shown in FIG. 1B.

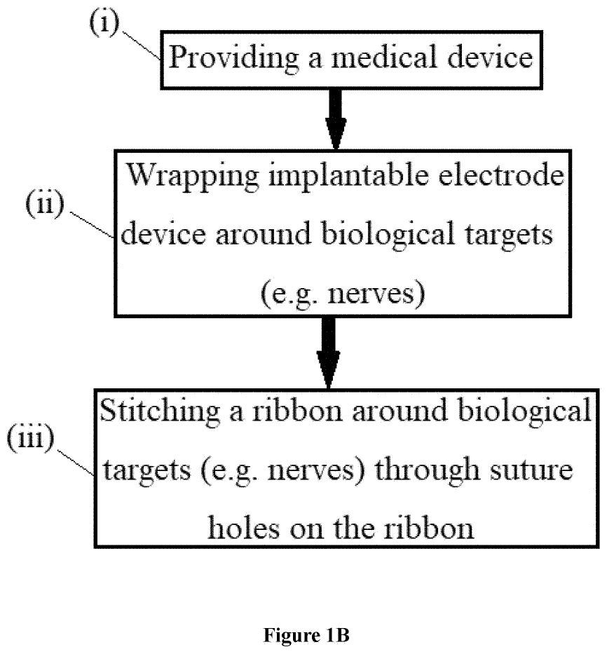

In various exemplary embodiments of the invention, an implantable lead is used to stimulate anterior and posterior spinal nerve components. FIG. 2 shows a flap wrapped around (partially or completely) an anterior nerve bundle and a posterior nerve bundle in accordance with an exemplary embodiment of the present invention. As shown in FIG. 2, the epineurium connecting the anterior nerve bundle 1 and posterior nerve bundle 2 is incised, separating the two nerve bundles from each other. Through the incision, flap 3 is wrapped around (partially or completely) the anterior nerve bundle 1 and posterior nerve bundle 2.

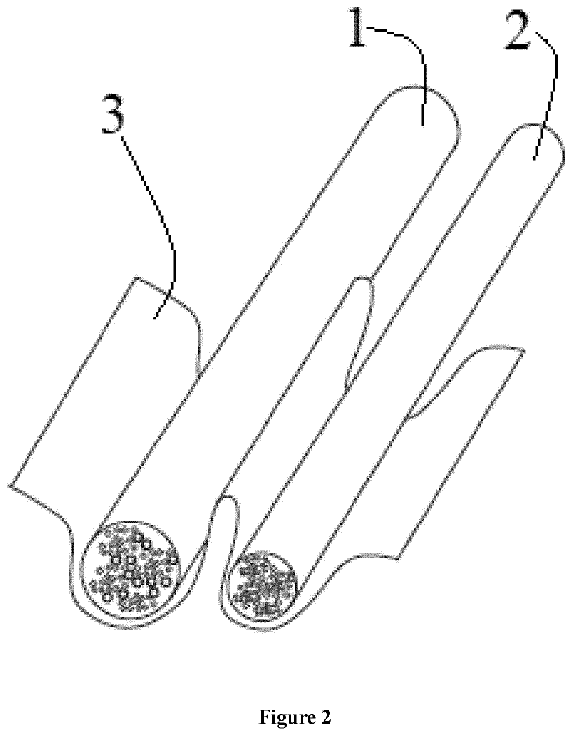

FIG. 3 illustrates electrodes on both sides of the flap are required to wrap around (partially or completely) an anterior nerve bundle and a posterior nerve bundle in accordance with an exemplary embodiment of the present invention. FIG. 3 shows another embodiment in which electrodes on both sides of flap 3 are required.

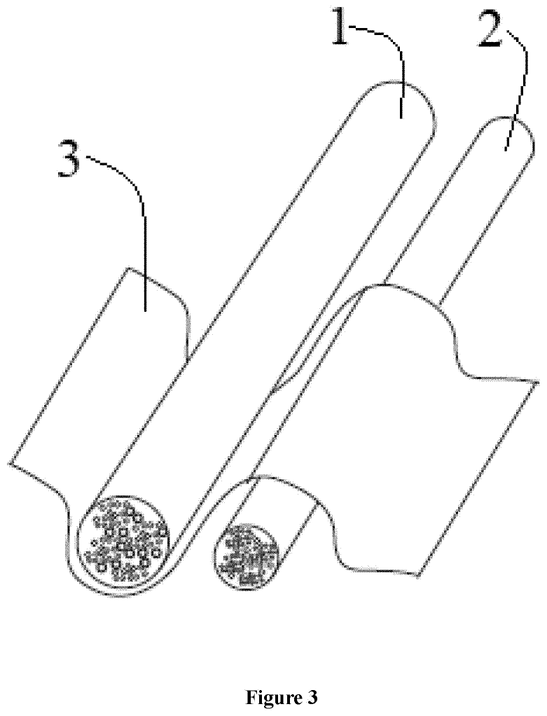

FIG. 4 demonstrates an implantable electrode device including microelectrode arrays and suture holes in accordance with an exemplary embodiment of the present invention. The electrodes in the implantable electrode device of the invention may include microelectrodes. The flap may include one, two or more electrode blocks, and each block comprises an array of microelectrodes. For example, the flap comprises two or more electrode blocks, and the blocks are arranged linearly along an elongation direction of the flap. One, two or more suture holes may be located in the proximity of each block. For instance, two or more suture holes may be arranged linearly along an elongation direction of the flap to form a hole-segment, and each electrode block is flanked by one or two of such hole-segments. As shown in FIG. 4, reference number 4 refers to a first electrode block (or “Stimulation Contacts I”). Reference number 5 refers to a second electrode block (or “Stimulation Contacts II”). Reference number 6 refers to suture holes arranged in three hole-segments Reference numbers 7a and 7b refer to connectors for wires. Stimulation Contact Blocks are not limited by individual contacts or stimulation blocks. Such a flap 3 is like a clothing belt with holes 6 but without a frame-style buckle or a plate-style buckle. Electrodes, electrode blocks, or microelectrode arrays (e.g. 4 and 5) can be placed around belt holes 6, preferably between belt holes 6.

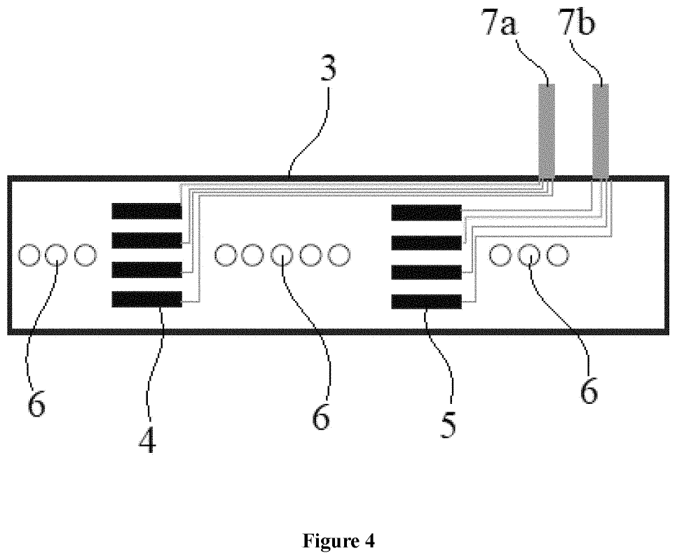

FIG. 5 depicts some details of a stimulation contact in accordance with an exemplary embodiment of the present invention. FIG. 5 shows some details of stimulation contact 4/5. Reference number 8a refers to a bridge (actual distance is very small but exaggerated here for illustrative purposes) between two microelectrodes 8b. Reference number 9 refers to a nerve. Thin film (flap) with small bridge 8 connection allows flexibility of the contact 4 or 5 to shape around the nerve 9.

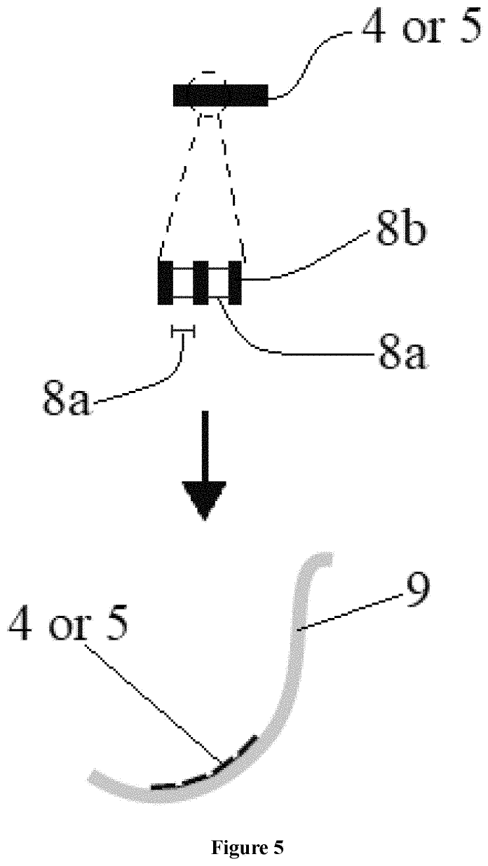

FIG. 6 illustrates the relationship between contacting areas “A”, “B” and “C” in the flap and corresponding contacting areas “A”, “B” and “C” in the nerves.

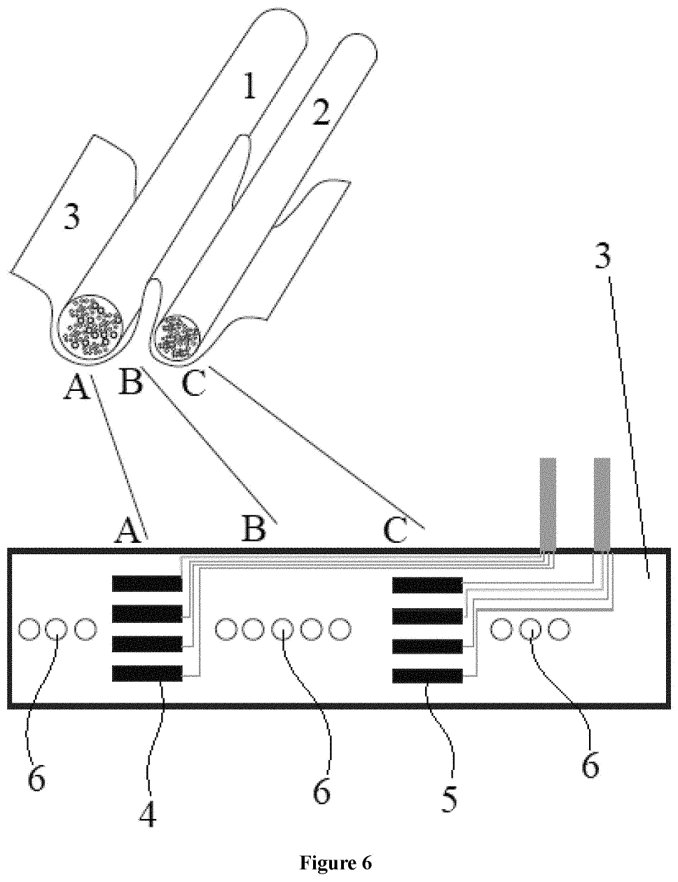

FIG. 7 schematically shows a manner how two flaps are wrapped around (partially or completely) an anterior nerve bundle and a posterior nerve bundle in accordance with an exemplary embodiment of the present invention. In FIG. 7, reference number 10 refers to a top flap Reference number 11 refers to a bottom flap. Reference number 12 refers to fixation points for suture, stitching, or clipping. Diameter d may be adjustable according to different nerve size.

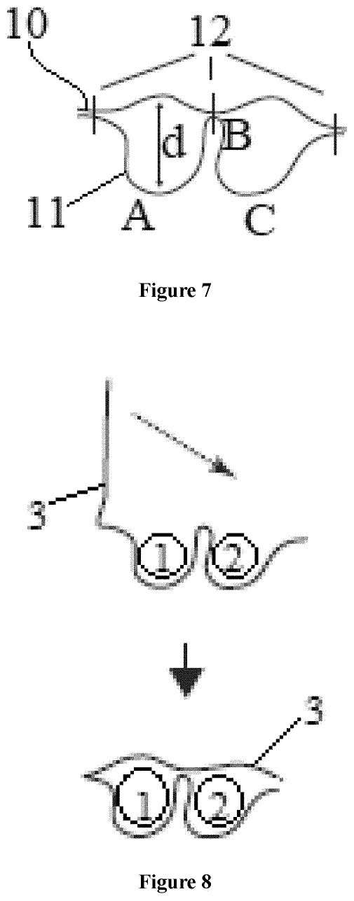

FIG. 8 schematically shows a manner how a single flap is wrapped around (partially or completely) an anterior nerve bundle and a posterior nerve bundle in accordance with an exemplary embodiment of the present invention. FIG. 8 shows another method to cover and fix the stimulation lead by flipping a single flap on top.

In the foregoing specification, embodiments of the present invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicant to be the scope of the invention, is the literal and equivalent scope of the set of claims that issue from this application, in the specific form in which such claims issue, including any subsequent correction.

1. An implantable electrode device comprising a flexible non-conductive flap, one or more electrodes integrated with the flap, and one or more wires embedded within the flap and connected to the one or more electrodes, wherein the implantable electrode device is configured to wrap around one, two or more biological targets, so that one of the electrodes can electrically and stably contact one of the biological targets; and wherein a center of the flap can be pulled, up between two biological targets such as two nerve segments, creating two circular like electrodes around each biological target such as nerve segment.

2. The implantable electrode device according to claim 1, wherein the flap has a front side and a back side, wherein one of the electrodes has only one flat contacting surface that can electrically contact one of the biological targets, and wherein the contacting surface is on the front side of the flap, or on the back side of the flap.

3. The implantable electrode device according to claim 1, wherein the flap has a front side and a back side, wherein one of the electrodes has only two flat contacting surfaces that can electrically contact one of the biological targets, and wherein one of the two contacting surfaces is on the front side of the flap, and another one is on the back side of the flap.

4. The implantable electrode device according to claim 1, wherein the flap comprises one or more suture holes for stitching the flap around the one, two or more biological targets.

5. The implantable electrode device according to claim 1, wherein the electrodes comprise microelectrodes, wherein the flap comprises one, two or more electrode blocks, and each block comprises an array of microelectrodes.

6. The implantable electrode device according to claim 5, wherein the flap comprises two or more electrode blocks, and the blocks are arranged linearly along an elongation direction of the flap.

7. The implantable electrode device according to claim 6, further comprising one, two or more suture holes located in the proximity of each block.

8. The implantable electrode device according to claim 7, wherein two or more suture holes are arranged linearly along an elongation direction of the flap to form a hole-segment, and each electrode block is flanked by one or two hole-segments.

9. A medical device or a medical system comprising the implantable electrode device of claim 1.

10. A diagnostic and/or therapeutic method comprising (i) providing a medical device/system according to claim 9; (ii) wrapping, the implantable electrode device according to claim 1 around one, two or more biological targets in a patient, so that one of the electrodes can electrically and stably contact one of the biological targets.

11. The method according to claim 10, wherein the biological targets include one, two or more nerves.

12. The method according to claim 11, wherein the flap comprises one or more suture holes for stitching the flap around the one or more nerves.

13. The method according to claim 12, further comprising stitching the flap around the one or more nerves through the one or more suture holes.
